(19) United States
(12) Patent Application Publication
(10) Pub. No.: US 2024/0399121 A1
(43) Pub. Date: Dec. 5, 2024

(54) MULTIPLE TOOL WIRELESS MICROCATHETER

(71) Applicant: Thomas Vacek, Elliston, VA (US)

(72) Inventor: Thomas Vacek, Elliston, VA (US)

(21) Appl. No.: 18/205,749

(22) Filed: Jun. 5, 2023

(51) Int. Cl. A61M 25/10; A61M 25/00; A61M 25/09

(52) U.S. Cl. CPC A61M 25/104; A61M 25/005; A61M 25/09; A61M 2025/0042

(57) ABSTRACT

A wireless microcatheter system for opening an occlusion in vascular beds is disclosed that does not require initial cross with a wire. The microcatheter system may include an tubular body having a body proximal end and a body distal end, wherein the body distal end has a most distal tip, the most distal tip having a length; an inflatable angioplasty balloon having a balloon proximal end and a balloon distal end; a guidewire having a proximal end and a distal end, where the distal end of the guidewire is threaded through an opening of the most distal tip; and a reciprocating actuator coupled with the guidewire to move the guidewire in various ways including reciprocating manner relative to the most distal tip in a jackhammer fashion, circular fashion, or to apply various types of energy including ultrasound or electrocautery to the plaque without requiring initial cross with a wire.

### BACKGROUND

### Technical Field

This disclosure relates to surgical tools. In particular, and without limitation the disclosure relates to an improved microcatheter for opening occlusions in a vascular bed of a patient.

Conventional microcatheters are useful for opening chronic total occlusions in multiple vascular beds. However, conventional microcatheters are limited in the types of tool ends or treatment procedures for addressing plaque in blood vessels. Also, some applications of conventional microcatheters to scrape or poke at caps of plaque in vessels may be ill-advised or even dangerous when not stabilized as is the case with this one.

Therefore, a need exists for an improved microcatheter fitted with multiple tool variations including a jackhammer motion for addressing plaque occlusions in vascular beds. This allows treatment of the cap of a plaque without the need to cross initially with a wire, which sometimes is not feasible and can keep one from having a successful intervention.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### SUMMARY

In an aspect, a microcatheter system for opening an occlusion in vascular beds is disclosed. The microcatheter system may include an elongate tubular body having a body proximal end and a body distal end, wherein the body distal end has a most distal tip, the most distal tip having a length; an inflatable angioplasty balloon having a balloon proximal end and a balloon distal end, wherein the balloon proximal end is attached to the length of the most distal tip at a first position and the balloon distal end is attached to the length of the most distal tip at a second position; and guidewire having a proximal end and a distal end, where the distal end of the guidewire is configured to be threaded through an opening of the most distal tip. Once the microcatheter is delivered over the wire, then wire is removed and alternate wires may be delivered that contain specialized tips used to treat the plaque and the distal end of the guidewire comprises a surgical tool locus for delivering a vascular occlusion tool to the occlusion.

In an aspect, method for opening an occlusion in a vascular bed is disclosed. The method may include inserting a microcatheter into a blood vessel in a body, the microcatheter comprising an elongate tubular body having a body proximal end and a body distal end, wherein the body distal end has a most distal tip, the most distal tip having a length; and an inflatable angioplasty balloon having a balloon proximal end and a balloon distal end, where the balloon proximal end is attached to the length of the most distal tip at a first position and the balloon distal end is attached to the length of the most distal tip at a second position; upon reaching a treatment location for the occlusion within the blood vessel, inflating the inflatable angioplasty balloon to secure the microcatheter in a position relative to an inner wall of the blood vessel such that the microcatheter has substantially no motion relative to the inner wall of the blood vessel; and inserting a guidewire having a proximal end and a distal end into an inner chamber of the microcatheter, where the distal end of the guidewire is configured to be threaded through an opening of the most distal tip and the distal end of the guidewire comprises a surgical tool locus.

Other systems, methods, features and advantages of the disclosure will be, or will become, apparent to one with skill in the art upon examination of the following figures and detailed description. It is intended that all such additional systems, methods, features and advantages be included within this description, be within the scope of the disclosure, and be protected by the following claims.

### BRIEF DESCRIPTION OF THE DRAWINGS

The disclosure can be better understood with reference to the following drawings and description. The components in the figures are not necessarily to scale, emphasis instead being placed upon illustrating the principles of the disclosure. Moreover, in the figures, like referenced numerals designate corresponding parts throughout the different views.

FIG. 1 is a view of an “over-the-wire” microcatheter system according to an aspect of the disclosure.

FIG. 2 is a view of a “rapid exchange” microcatheter system according to an aspect of the disclosure.

FIG. 3A, FIG. 3B and FIG. 3C are views of vascular occlusion tools according to an aspect of the disclosure.

FIG. 4 is a view of an “over-the-wire” (OTW) microcatheter system according to an aspect of the disclosure.

FIG. 5 is a view of a third aspect of a microcatheter device according to an aspect of the disclosure.

FIG. 6 is a flow chart of acts taken in a process to open an occlusion in a vascular bed according to an aspect of the disclosure.

### DETAILED DESCRIPTION

An improved microcatheter for opening occlusions in a vascular bed is disclosed. In an aspect, a surface of the disclosed microcatheter may be reinforced or braided to resist pressure from an air lumen or angioplasty balloon surrounding the column body of the microcatheter. The microcatheter system may be delivered OTW (over the wire), or in RX (rapid exchange).

In an aspect, once delivered to the desired site within the blood vessel, the disclosed microcatheter will have the angioplasty balloon inflated and a stiffer guidewire will be placed in the microcatheter. The stiffer guidewire may be inserted into an electronic device whereby the guidewire traverses in a reciprocating motion forward and backward, functioning essentially as a jackhammer to address a plaque cap on the occlusion of the blood vessel. The disclosed microcatheter system would also allow for one to take the stiff guidewire of the catheter and manually poke at the calcified plaque.

In an aspect, the disclosed microcatheter is configurable to provide a set of “wireless power tools kit” for opening chronic total occlusions in multiple vascular beds, but especially peripheral vascular space and coronary vascular space with sizing of 0.014, 0.18, 0.035 microcatheter sizing. These tools will be especially important for the most challenging blunted caps but may be utilized for tapered caps as well in difficult to cross regions.

In an aspect, the disclosed microcatheter will be a balloon-stabilized microcatheter, stabilized by an inflatable angioplasty balloon in communication with the microcatheter body, including an electronic device that can shift a guidewire contained within the microcatheter forward and backward. The electronic device may also spin the guidewire. In an aspect, the electronic device may emit electrical cautery impulse to address the vascular occlusion.

In an aspect, the disclosed microcatheter may configured as a “jackhammer” for addressing a vascular occlusion. The guidewire contained in and directed through the microcatheter may be capable of reciprocating motion forward and backward in a longitudinal direction relative to a length of the microcatheter. The jackhammer motion of the guidewire may be directed by a mechanical device, an electronic device or manually directed.

In an aspect, the disclosed microcatheter may include a drill bit tool, a wire with a specialized tip for drilling into plaque caps within a blood vessel. The drill bit tool wire may be used with the mechanical box to turn it clockwise or counterclockwise or can be turned manually.

In an aspect, the disclosed microcatheter may include a “sander,” which is a wire with an abrasive blunted tip that also extends through the microcatheter and is stabilized through the microcatheter with balloons attached outside the microcatheter column. A sanding action by the sander is activated by rotating the specialized wire tip clockwise or counterclockwise. In an aspect, the rotation can be accomplished by the mechanical box or also manually.

In an aspect, the disclosed microcatheter may include a welding tool, which applies a cauterizing impulse to a plaque cap of an occlusion. The safety of the welding tool is enhanced by the balloon stabilization of the microcatheter.

In an aspect, the disclosed microcatheter may include a lithotripsy energy emitter (lithotripter). The lithotripter will be at end of the wire at will be utilized to help to break up calcium which is a component of atherosclerotic plaque or lesion that will help to facilitate further wire entry distally.

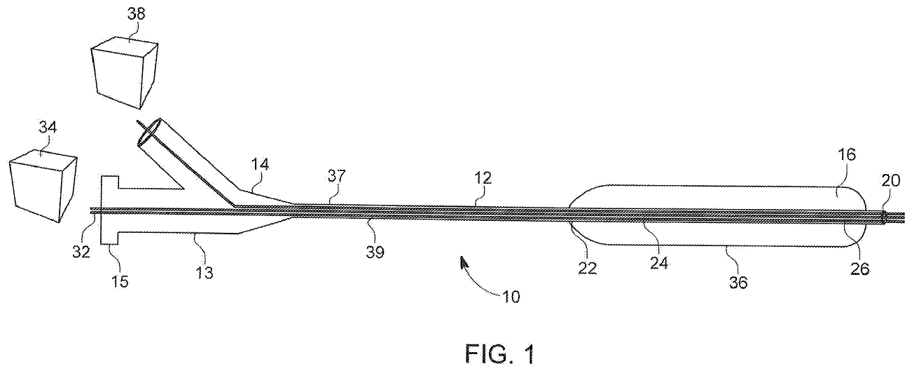

FIG. 1 is a schematic illustration of an “over-the-wire” (OTW) microcatheter system 10 in accordance with aspects of the present disclosure. The OTW microcatheter system 10 has an elongate tubular body 12 having a body proximal end 14 and a body distal end 16. The body proximal end 14 is in communication to an introduction port 13 which can be used for introduction of a guidewire into the microcatheter 10. The introduction port 13 may include a guidewire port 15 for introduction of a guidewire 32 therethrough. A tip portion 22 is positioned distal to body distal end 16. The tip portion 22 may be continuous with elongate tubular body 12 or may be separated therefrom, as will be described in further detail below. An outlet port 20 is positioned at body distal end 16, proximal to tip portion 22. The outlet port 20 may comprise one or multiple openings. The tip portion 22 has a tip portion proximal end 24, and a tip portion distal end 26 having a most distal tip. The tip portion 22 is sized to snugly fit a guidewire 32 therethrough along the entire length of tip portion 22. It should be readily apparent that tip portion 22 may have other diameters, provided that a guidewire 32 fits snugly inside. The tight fit for the guidewire provides for minimization of the distal portion of microcatheter system 10 and enhanced pushability and maneuverability of the guidewire. A guidewire 32 is shown positioned within microcatheter 10 and extending through tip portion 22.

In an aspect, the microcatheter system 10 may include an actuating device 34 in communication with the guidewire 32 at the proximal end of the guidewire 32. The actuating device 34 engages with the guidewire 32 to move the guidewire 32 relative to the most distal tip. In an aspect, the actuating device 34, the actuating device 34 moves the guidewire 32 in a reciprocating manner backwards and forwards relative to the most distal tip, effecting a “Jackhammer” method of treating a plaque cap of the occlusion. In an aspect, the actuating device 34 moves the guidewire 32 in a circular motion relative to a longitudinal axis of the most distal tip. In an aspect, the actuating device 34 transmits a cautery impulse to the distal end of the guidewire 32.

At the body distal end 16 of the OTW microcatheter system 10 may be a 1.0-12 mm diameter angioplasty balloon 36 depending on which vascular bed is being worked in but is not limited to these sizes if utilized in larger vascular beds. The angioplasty balloon 36 will be compliant or semi-compliant and able to be inflated at a range of sizes in accordance with how many atmospheres pressure are placed in balloon. There is an air lumen 37 that connects into where the angioplasty balloon 36 is inflated so as to modulate the pressure within the angioplasty balloon 36 to inflate or deflate using an indeflator device 38 as conventionally used with a microcatheter system 10.

In an aspect, for the OTW microcatheter system 10, the OTW microcatheter system 10 is placed using the guidewire 32 through the microcatheter lumen 39 and then the guidewire 32 is subsequently removed, the angioplasty balloon 36 can be inflated, and tools, such as vascular occlusion tools, can be inserted through the microcatheter lumen 37 of the microcatheter system 10 thereafter to be controlled by the actuator device 34.

In an aspect, the elongate tubular body 12 may have various aspects. The elongate tubular body 12 may have a surface 13 that is braided or reinforced to resist pressure from an angioplasty balloon 36. The elongate tubular body 12 may accommodate larger diameter guidewires 32 that may be substituted for different functionality employed with the microcatheter system 10. In all of the aspects, guidewire 32 is positioned through at least tip portion 22.

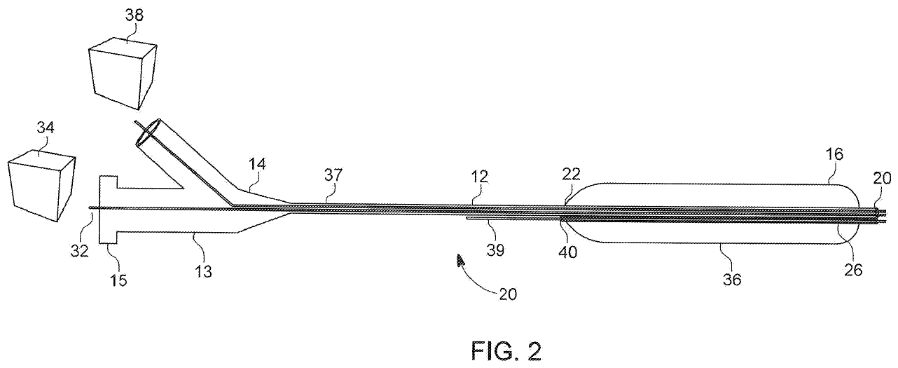

In an aspect, the inflatable angioplasty balloon 36 may be a compliant or semi-compliant balloon, both types of which are commonly known in the art. The aspect shown in FIG. 2, when used with a semi-compliant balloon in particular, is suitable, for example, for treating a chronic total occlusion (CTO). In a case of CTO, the inflatable angioplasty balloon 36 can be used to break up the lesion and then advance guidewire 32 through the lesion.

FIG. 2 is a schematic illustration of a “rapid exchange” (RX) microcatheter system 20 in accordance with aspects of the present disclosure. The RX microcatheter system 20 in FIG. 2 reproduces the same elements with like reference numerals from FIG. 1 for the OTW microcatheter system 10. In an RX microcatheter system 20, there is additionally an RX port 40 positioned proximal to the tip portion 22. The RX port 40 presents another opening port to insert plaque modifying tools such as vascular occlusion tools, described herein, into the RX microcatheter system 40 to treat vascular occlusions in a vascular bed. In an aspect, a wire threaded through the RX port 40 may include a surgical tool locus for receiving vascular occlusion tools, or the wire itself is capped with one of various vascular occlusion tools.

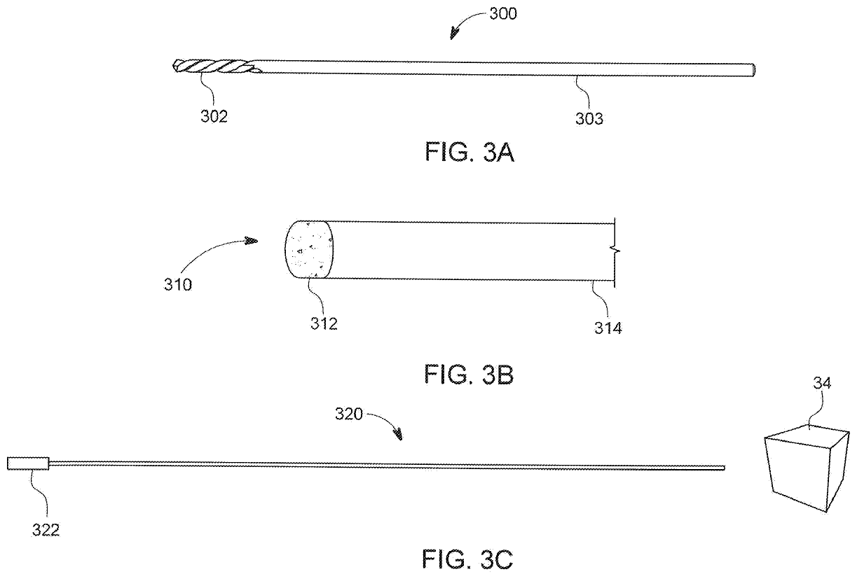

FIG. 3A, FIG. 3B and FIG. 3C are views of vascular occlusion tools that may be used with the microcatheter system 10 according to the disclosure. FIG. 3A is a view of a microdrill bit 300 for addressing the occlusion within the blood vessel. The microdrill bit 300 includes a microdrill tip 302 at the distal end of the guidewire 303 inserted through the distal tip portion distal end 26 of the elongate tubular body 12 of the microcatheter system 10. The microdrill bit 302 may in an aspect be connected to a surgical tool locus for receiving vascular occlusion tools at the distal end of the guidewire 303. The microdrill tip 302 may be of different lengths and have different numbers of grooves along the microdrill tip 302 as applicable for the application. The microdrill tip 302 may be of a surgically compatible material, such as surgical steel, to be of use in the human body.

The microdrill bit 300, through the guidewire 303 on which the microdrill bit 300 caps an end of the guidewire 303, may be rotated by the actuating device 34 so as to drill away at a plaque cap of the occlusion or other tissue to be addressed in the treatment. In an aspect, the microdrill bit 300 may be rotated with an electronic device, a mechanical device or a manually driven technique.

FIG. 3B is a view of a sanding end face 310 for addressing the occlusion within the blood vessel. The sanding end face 310 includes a sanding tip 312 at the distal end of the guidewire 314 inserted through the distal tip portion distal end 26 of the elongate tubular body 12 of the microcatheter system 10. The sanding tip 312 may comprise a blunted end of the guidewire 314, with a coarse surface and which fits through the microcatheter system 10. The sanding end face 310 will be of necessity of a diameter to allow it to fit into the introduction port 13. The sanding end face 310 may in an aspect be connected to a surgical tool locus for receiving vascular occlusion tools at the distal end of the guidewire 303.

The sanding end face 310, through the guidewire 303, may be rotated by the actuating device 34 so as to sand or grind away at a plaque cap of the occlusion or other tissue to be addressed in the treatment. In an aspect, the sanding end face 310 may be rotated with an electronic device, a mechanical device or a manually driven technique.

FIG. 3C is a view of a lithotripsy energy emitter (lithotripter) 322 configured to break up calcium deposits inside a vascular bed with a series of focused acoustic shock waves delivered through the lithotripter 322. The lithotripter 322 is at a distal end of the delivery wire 320, such as a guidewire described above. At a proximal end, the delivery wire 320 connects the lithotripter 322 to a utility box 34. The lithotripter 322 is placed proximate to the treatment area, such as a calcium deposit, which needs to be broken up with focused acoustic waves emitted from the lithotripter 322.

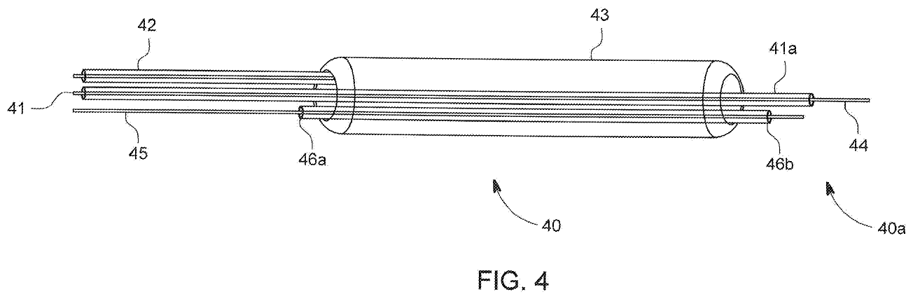

FIG. 4 is a close-up schematic view of an outlet port 40a for a microcatheter system 40 as illustrated in FIG. 1 and FIG. 2. A microcatheter 41 extends along the longitudinal length of the microcatheter system 40. An air lumen 42 is disposed upon an outer surface of the microcatheter 41, extending toward a distal end 41a of the microcatheter 41. An inflatable angioplasty balloon 43 is disposed near the distal end 41a of the microcatheter 41, where the inflatable angioplasty balloon 43 is inflated through the air lumen 42 from a indeflator apparatus (not illustrated). In an aspect, wire tools 44, such as the vascular occlusion tools described with reference to FIGS. 3A-3C, may be inserted through the microcatheter 41 and exit at the distal end 41a of the microcatheter 41. In an aspect, a standard wire 45 may be inserted into an RX entry port 46a and exit from an RX exit port 46b, to deliver vascular occlusion tools, such as those described with reference to FIGS. 3A-3C, over the standard wire 45. In an aspect, the standard wire 45 may include a surgical tool locus for receiving vascular occlusion tools, or the standard wire 45 itself is capped with one of various vascular occlusion tools.

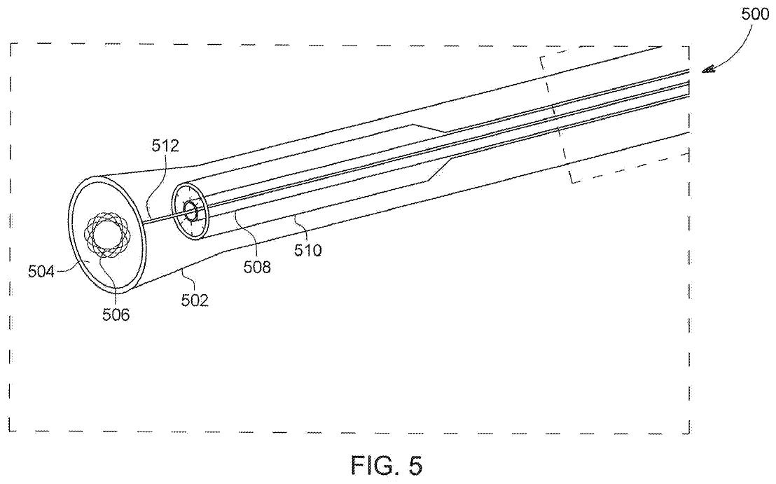

FIG. 5 is a view of an aspect of a microcatheter system 500, according to the disclosure. The microcatheter system 500 is illustrated in FIGS. 1-4 within a blood vessel 502, in which an occlusion 504 having a plaque cap 506 to be addressed is present. The microcatheter system 500 includes a microcatheter 508 which may be surrounded by an air lumen 510, which may be in the form of an inflatable angioplasty balloon. Within the microcatheter 508 is a guidewire 512 threaded throughout the length longitudinally of the microcatheter 508.

When the microcatheter system is positioned at the locus of treatment proximate to the plaque cap 506 or other tissue to be addressed, the air lumen 510 or balloon is inflated to secure the microcatheter 508 in a position relative to an inner wall of the blood vessel 502 such that the microcatheter 508 has substantially no motion relative to the inner wall of the blood vessel 502.

The guidewire 512 may be replaced with a wire of similar diameter or wire strength to treat the occlusion with a pointed tip in a “jackhammer” manner. The guidewire 512 is in communication with an actuating device (element 34 in FIG. 1) or a reciprocating motion actuator coupled with the guidewire 512 at the proximal end of the guidewire 512 and configured to move the guidewire 512 in a reciprocating manner relative to the most distal tip of the microcatheter 508. In this manner, the guidewire 512 acts as a “jackhammer” to poke at the plaque cap 506 or other tissue.

In an aspect, the guidewire 512 may include a surgical tool locus for receiving vascular occlusion tools, or the guidewire 512 itself is capped with one of various vascular occlusion tools.

In an aspect, the guidewire 512 may include a microdrill bit as illustrated in FIG. 3A for drilling at the plaque cap 506 or other tissue to be treated. The actuating device coupled with the guidewire 512 may move the guidewire 512 in a circular motion relative to a longitudinal axis of the most distal tip of the microcatheter 508 to rotate the microdrill bit on the distal end of the guidewire 512.

In an aspect, the guidewire 512 may include a sander end face as illustrated in FIG. 3B for sanding or grinding the plaque cap 506 or other tissue to be treated. The actuating device coupled with the guidewire 512 may move the guidewire 512 in a circular motion relative to a longitudinal axis of the most distal tip of the microcatheter 508 to rotate the sander end face on the distal end of the guidewire 512.

In an aspect, the actuating device may transmit a cautery impulse to the distal end of the guidewire 512. In an aspect, the vascular occlusion tool may include a welding tip configured to channel the cautery impulse for addressing the occlusion. The welding tip may merely be the distal end of the guidewire 512 in an aspect, the welding tip may be a vascular occlusion tool coupled to the distal end of the guidewire 512 via a surgical tool locus. In an aspect, any of the aforementioned vascular occlusion tools may be used as a cautery tool, where the cautery impulse is directed through the tip of the vascular occlusion tool to the area to be treated.

In an aspect, the guidewire 512 mar include a lithotripsy energy emitter (lithotripter) as illustrated and described in relation to FIG. 3C for use in to break up calcium deposits in a vascular bed with a series of focused acoustic shock waves generated by the lithotripter.

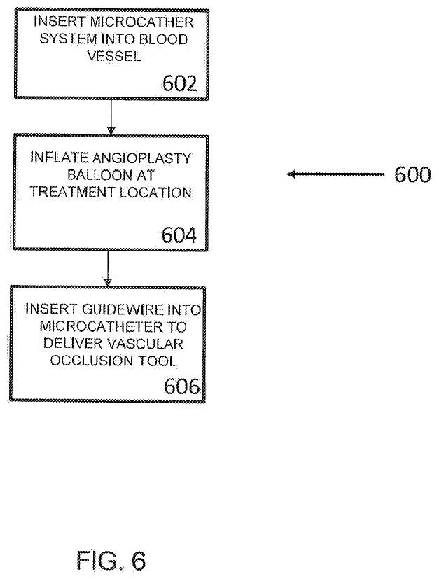

FIG. 6 is a flow chart 600 illustrating acts that may be taken to use the disclosed microcatheter system to open occlusions in a vascular bed. The microcatheter system may be any of the aspects of the microcatheter system described in relation to FIGS. 1-5. Control begins at act 602.

At act 602, a microcatheter is inserted into a blood vessel in a body. The microcatheter may consist of an elongate tubular body having a body proximal end and a body distal end, where the body distal end has a most distal tip, the most distal tip having a length.

The microcatheter may be in communication with an inflatable angioplasty balloon having a balloon proximal end and a balloon distal end, where the balloon proximal end is attached to the length of the most distal tip at a first position and the balloon distal end is attached to the length of the most distal tip at a second position.

At act 604, upon reaching a treatment location for the occlusion within the blood vessel, the inflatable angioplasty balloon is inflated to secure the microcatheter in a position relative to an inner wall of the blood vessel such that the microcatheter has substantially no motion relative to the inner wall of the blood vessel. This may serve to secure the microcatheter in place while treatment is performed on the occlusion. Movement of the microcatheter may damage surrounding tissue and/or the blood vessel walls, resulting in trauma to the tissue, possible bleeding out of the blood vessel or rupturing plaque unnecessarily.

At act 606, a guidewire is inserted into the microcatheter and threaded through the microcatheter via an inner chamber along the microcatheter. The guidewire may be delivered “over the wire” (OTW) and not “rapid exchange” (RX). The guidewire has a proximal end and a distal end into an inner chamber of the microcatheter. The distal end of the guidewire is configured to be threaded through an opening of the most distal tip and the distal end of the guidewire comprises a surgical tool locus for delivering a vascular occlusion tool to the occlusion.

Once the guidewire is inserted into the microcatheter, the guidewire may be delivered to the occlusion to be treated. The guidewire may be configured to receive surgical tools through an interface, such as the surgical tool receptable, on the most distal tip of the guidewire. In an aspect, the guidewire may be removed to replace the guidewire with a surgical tool already placed on the most distal tip.

In an aspect, an actuating device may be positioned in communication with the guidewire at the proximal end of the guidewire. The actuating device engages with the guidewire to move the guidewire relative to the most distal tip. The actuating device may be an electronic device with an actuating motor. In an aspect, the actuating device may be omitted and the guidewire is moved manually by the operator of the microcatheter. moving, by the actuating device, the guidewire in a reciprocating manner backwards and forwards relative to the most distal tip. In such a manner, the guidewire may function as a jackhammer to address and poke at a plaque cap of the occlusion to open the occlusion. In an aspect, the actuating device may include a reciprocating motion actuator coupled with the guidewire at the proximal end of the guidewire and configured to move the guidewire in a reciprocating manner relative to the most distal tip.

In an aspect, the actuating device may move the guidewire in a circular motion relative to a longitudinal axis of the most distal tip. With such motion, the vascular occlusion tool may include a microdrill bit for addressing the occlusion, operating in a manner similar to a conventional drill bit to remove portions of the plaque cap of the occlusion. In an aspect, the vascular occlusion tool may include a sanding end face for addressing the occlusion. The sanding tool end face may include an abrasive material use to “sand” or grind away material on the plaque cap of the occlusion.

In an aspect, the actuating device may transmit a cautery impulse to the distal end of the guidewire. In an aspect, the vascular occlusion tool may include a welding tip configured to channel the cautery impulse for addressing the occlusion.

In an aspect, the guidewire may be removed and replaced with a wire with a tool-accepting end to engage with vascular occlusion tools as described herein. The wire with the tool-accepting end may be inserted in an RX port in communication with the microcatheter system to deliver vascular occlusion tools through the RX port to address a vascular occlusion at a treatment site through an RX exit port in communication with the microcatheter.

It is to be understood by one of skill in the art that the disclosed microcatheter system may be composed of surgically compatible materials for use inside a human body. It is to be understood that the materials may be appropriate nature to allow flexible and dynamic interaction with tissue in the human body, particularly, but without limitation, plaque occlusions in a vascular bed within the human body. Other features, components, additions and techniques of use of the microcatheter system may be contemplated without deviating from the scope of the disclosure. The microcatheter system may include, for example, fiber optic camera and control devices; solution dispensers for injecting a medical solution into a human blood vessel.

When introducing elements of the present disclosure, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary aspects described above should not be used to limit the scope of the disclosure. Accordingly, the disclosure is not to be restricted except in light of the attached claims and their equivalents.

What is claimed is:

1. A microcatheter system for opening an occlusion in a vascular bed, comprising:
an elongate tubular body having a body proximal end and a body distal end, where the body distal end has a most distal tip, the most distal tip having a length;
an inflatable angioplasty balloon having a balloon proximal end and a balloon distal end, wherein the balloon proximal end is attached to the length of the most distal tip at a first position and the balloon distal end is attached to the length of the most distal tip at a second position; and
a guidewire having a proximal end and a distal end, where the distal end of the guidewire is configured to be threaded through an opening of the most distal tip and the distal end of the guidewire comprises a surgical tool locus for delivering a vascular occlusion tool to the occlusion.

2. The microcatheter system of claim 1, where an external surface of the elongate tubular body comprises a reinforced material or a braided material.

3. The microcatheter system of claim 1, where the microcatheter system includes an elongate tubular body having a body proximal end and a body distal end, where the elongate tubular body comprises an outlet port at the body distal end, a tip portion having a tip portion proximal end and a tip portion distal end, the tip portion proximal end positioned distal to the outlet port of the elongate tubular body, and where the inflatable angioplasty balloon is positioned on the tip portion.

4. The microcatheter system of claim 1, further comprising an actuating device in communication with the guidewire at the proximal end of the guidewire, the actuating device engaging with the guidewire to move the guidewire relative to the most distal tip.

5. The microcatheter system of claim 4, where the actuating device moves the guidewire in a reciprocating manner backwards and forwards relative to the most distal tip.

6. The microcatheter system of claim 4, where the actuating device moves the guidewire in a circular motion relative to a longitudinal axis of the most distal tip.

7. The microcatheter system of claim 4, where the actuating device transmits a cautery impulse to the distal end of the guidewire.

8. The microcatheter system of claim 6, where the vascular occlusion tool comprises a microdrill bit for addressing the occlusion.

9. The microcatheter system of claim 6, where the vascular occlusion tool comprises a sanding end face for addressing the occlusion.

10. The microcatheter system of claim 7, where the vascular occlusion tool comprises a welding tip configured to channel the cautery impulse for addressing the occlusion.

11. The microcatheter system of claim 1, where the vascular occlusion tool comprises a lithotripter configured to treat calcium in atherosclerotic lesions present in the human body.

12. A method for opening an occlusion in a vascular bed, the method comprising:
inserting a microcatheter into a blood vessel in a body, the microcatheter comprising an elongate tubular body having a body proximal end and a body distal end, wherein the body distal end has a most distal tip, the most distal tip having a length; and an inflatable angioplasty balloon having a balloon proximal end and a balloon distal end, where the balloon proximal end is attached to the length of the most distal tip at a first position and the balloon distal end is attached to the length of the most distal tip at a second position;
upon reaching a treatment location for the occlusion within the blood vessel, inflating the inflatable angioplasty balloon to secure the microcatheter in a position relative to an inner wall of the blood vessel such that the microcatheter has substantially no motion relative to the inner wall of the blood vessel; and
inserting a guidewire having a proximal end and a distal end into an inner chamber of the microcatheter, where the distal end of the guidewire is configured to be threaded through an opening of the most distal tip and the distal end of the guidewire comprises a surgical tool locus for delivering a vascular occlusion tool to the occlusion.

13. The method of claim 12, further comprising positioning an actuating device in communication with the guidewire at the proximal end of the guidewire, the actuating device engaging with the guidewire to move the guidewire relative to the most distal tip.

14. The method of claim 13, further comprising moving, by the actuating device, the guidewire in a reciprocating manner backwards and forwards relative to the most distal tip.

15. The method of claim 13, further comprising moving, by the actuating device, the guidewire in a circular motion relative to a longitudinal axis of the most distal tip.

16. The method of claim 13, further comprising transmitting, by the actuating device, a cautery impulse to the distal end of the guidewire.

17. The method of claim 15, where the vascular occlusion tool comprises a microdrill bit for addressing the occlusion.

18. The method of claim 15, where the vascular occlusion tool comprises a sanding end face for addressing the occlusion.

19. The method of claim 16, where the vascular occlusion tool comprises a welding tip configured to channel the cautery impulse for addressing the occlusion.

20. A microcatheter system for opening an occlusion in vascular beds, comprising:
an elongate tubular body having a body proximal end and a body distal end, wherein the body distal end has a most distal tip, the most distal tip having a length;
an inflatable angioplasty balloon having a balloon proximal end and a balloon distal end, wherein the balloon proximal end is attached to the length of the most distal tip at a first position and the balloon distal end is attached to the length of the most distal tip at a second position;
a guidewire having a proximal end and a distal end, where the distal end of the guidewire is configured to be threaded through an opening of the most distal tip; and
a reciprocating motion actuator coupled with the guidewire at the proximal end of the guidewire and configured to move the guidewire in a reciprocating manner relative to the most distal tip.
